(19) United States
(12) Patent Application Publication
(10) Pub. No.: US 2025/0032771 A1
(43) Pub. Date: Jan. 30, 2025

(54) INTRAVASCULAR BLOOD PUMPS AND EXPANDABLE SCAFFOLDS WITH STIFFENING MEMBERS

(71) Applicant: Shifamed Holdings, LLC, Campbell, CA (US)

(72) Inventor: Daniel HILDEBRAND, Santa Cruz, CA (US)

(21) Appl. No.: 18/710,551

(22) PCT Filed: Nov. 16, 2022
(86) PCT No.: PCT/US2022/079950

§ 371 Date: May 15, 2024

Related U.S. Application Data
(60) Provisional application No. 63/264,133, filed on Nov. 16, 2021.

(51) Int. Cl. A61M 60/13; A61M 60/216; A61M 60/408; A61M 60/857

(52) U.S. Cl. CPC A61M 60/13; A61M 60/216; A61M 60/408; A61M 60/857

(57) ABSTRACT

Catheter blood pumps that include an expandable blood conduit and at least one impeller. Tension members extending from the blood conduit are associated with increasing a stiffness of an impeller region housing the impeller, thereby increasing the impeller region's resistance to deflection, and maintaining clearance for the impeller. In some cases, a tension control element is used to control a degree of tension placed on the tension members. The tension control element may be used to increase or decrease the stiffness of the impeller region and/or modulate the size of a blood inlet or outlet of the blood conduit.

### CROSS REFERENCE TO RELATED APPLICATIONS

This application claims priority to U.S. Provisional Application No. 63/264, 133, filed on Nov. 16, 2021, and entitled “INTRAVASCULAR BLOOD PUMPS AND EXPANDABLE SCAFFOLDS WITH STIFFENING MEMBERS”, which is incorporated herein by reference in its entirety.

This application may be related to International Application No. PCT/US22/19187, filed on Mar. 7, 2022, published as WO 2022/187747, and entitled “INTRAVASCULAR BLOOD PUMPS AND PUMPS WITH EXPANDABLE SCAFFOLDS,” which is incorporated herein by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. Intra-aortic balloon pumps (IABP) are used to support circulatory function, such as treating heart failure patients. An IABP is typically placed within the aorta, and inflated and deflated in counter-pulsation fashion with the heart contractions, with one function being to provide additive support to the circulatory system. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or with pharmacological treatment.

More recently, minimally invasive rotary blood pumps have been developed, which are inserted into the body in connection with the cardiovascular system to pump arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient. The smallest rotary blood pumps currently available may be percutaneously inserted into the vasculature of a patient through an access sheath, thereby avoiding more extensive surgical intervention, or through a vascular access graft. One such device is a percutaneously inserted ventricular support device.

Although blood pumps exist, there is a need to provide additional improvements in the field of ventricular support devices and similar blood pumps for treating compromised cardiac blood flow.

### SUMMARY OF THE DISCLOSURE

The disclosure is related to intravascular blood pumps and their methods of manufacture and use. The blood pumps may include an expandable conduit that is sized and shaped to allow blood to flow therethrough when in an expanded state. The blood pumps may include one or more impellers that may be at least partially within the conduit and is/are configured to pump blood through the conduit. Described herein are features that may increase or decrease a lateral stiffness of at least part of the conduit. For example, increasing the lateral stiffness may reduce or prevent deformation (e.g., lateral deflection) of the conduit when the blood pump is within a patient's heart. In some cases, increasing the lateral stiffness of an impeller region of the conduit may provide clearance for the impeller(s) during operation. In some examples, the lateral stiffness may be tuned to a desired degree of stiffness.

One aspect of the disclosure is an intravascular blood pump, comprising: a collapsible blood conduit having an inner lumen for passing blood therethrough, wherein a proximal portion of the blood conduit includes an impeller region configured to accommodate at least a portion of an impeller therein; and a plurality of struts and one or more tension members extending proximally from the blood conduit and radially inward toward a shaft, wherein the plurality of struts are proximally coupled to the shaft at a first axial region along the shaft, and the one or more tension members is proximally coupled to the shaft at a second axial region along the shaft, wherein first axial region is at a different axial position along the shaft than the second axial region.

The plurality of struts and one or more tension members may be configured to provide the impeller region resistance to lateral deflection. The second axial region may extend further proximally along the shaft relative to the first axial region. The first axial region may extend further proximally along the shaft relative to the second axial region. At least a portion of the one or more tension members may be radially outside of the plurality of struts. The blood conduit may include an expandable scaffold comprising a pattern of axially and radially extending elements. The blood conduit may include a membrane that covers the expandable scaffold. The one or more tension members may be fixedly coupled to the shaft at the second axial region. The one or more tension members may be coupled to the shaft by a tension control element that is configured to move axially relative to the shaft, wherein axial movement of the tension control element adjusts an amount of tension placed on the one or more tension members and an amount of lateral stiffness imparted to the impeller region of the blood conduit. The tension control element may include a locking feature configured to lock an axial position of the tension control element relative to the shaft. The one or more tension members may include a plurality of tension members. The plurality of tension members may be equally radially distributed around a proximal end of the blood conduit. The plurality of tension members may be equally radially distributed the shaft. The plurality of struts may be equally radially distributed around a proximal end of the blood conduit. The plurality of struts may be equally radially distributed around the shaft. The plurality of struts may be coupled to a hub of the shaft. The one or more tension members may be coupled to a hub of the shaft. The plurality of struts may be coupled at the same axial position along the shaft. The plurality of struts may be coupled along at different axial positions along the shaft. The one or more tension members may include a plurality of tension members, wherein the plurality of tension members may be coupled at the same axial position along the shaft. The one or more tension members may include a plurality of tension members, wherein the plurality of tension members are coupled at different positions along the shaft. The plurality of struts may form a first conical structure having a large end pointing in a distal direction toward the blood conduit; and the one or more tension members may include a plurality of tension members that forms a second conical structure having a large end pointing in the distal direction toward the blood conduit. The plurality of struts may curve radially inward toward the shaft. The one or more tension members may be substantially straight. Each of the plurality of struts may be connected with at least one of the one or more tension members at a junction region. The junction region may be at a proximal end of the blood conduit. Each of the plurality of struts may be connected to a first tension member and a second tension member at or near the junction region. A span angle between the first tension member and the second tension member may be less than or equal to 75 degrees. A span angle between the first tension member and the second tension member may be less than or equal to 60 degrees. A tension member angle plus a strut angle may be less than 90 degrees, wherein the tension member angle is measured between a tension member line and a strut line, wherein the strut angle is measured between a strut line and a diameter line of the blood conduit, wherein the tension member line is measured from a point of connection of a tension member to the diameter line of the blood conduit to a point of connection of the tension member along a diameter line of shaft, wherein the strut line is measured from a point of connection of a strut along the diameter line of blood conduit to a point of connection of the strut along the diameter line of the shaft. The intravascular blood pump may further comprise the impeller within the impeller region of the blood conduit. The intravascular blood pump may further comprise a second impeller within the blood conduit.

One aspect of the disclosure is a method of using an intravascular blood pump, the method comprising: delivering the blood pump towards a heart while a blood conduit of the blood pump is in a collapsed state; expanding a blood conduit of the blood pump within the heart, the expanded blood conduit defining an inner lumen, wherein a proximal portion of the blood conduit includes an impeller region having at least a portion of an impeller therein; and causing the impeller to rotate and pump blood through the inner lumen of the blood conduit and out of a proximal end of the blood conduit, wherein the blood pump includes a plurality of struts and one or more tension members configured to provide the impeller region resistance to deflection, the plurality of struts and the one or more tension members extending proximally from the blood conduit and radially inward toward a shaft, wherein the plurality of struts are proximally coupled to the shaft at a first axial region along the shaft, and the one or more tension members is proximally coupled to the shaft at a second axial region along the shaft, wherein first axial region is at a different axial position along the shaft than the second axial region.

The blood may be pumped into a distal opening of the blood conduit and out of a proximal opening of the blood conduit. The second axial region may extend further proximally along the shaft relative to the first axial region. The method may further comprise collapsing the blood conduit into a catheter and removing the blood pump from the heart. The one or more tension members may be coupled to the shaft via a tension control element, the method further comprising translating the tension control element axially along the shaft to control a degree of tension placed on the tension members, higher tension on the tension members is associated with a higher stiffness of the impeller region. The method may further comprise locking the tension control element at an axial location relative to the shaft. The tension control element may be adjusted prior to delivering the blood pump to the heart. The tension control element may be adjusted while the blood pump is within the heart. The blood may be pumped into a distal opening of the blood conduit and out of a proximal opening of the blood conduit, wherein increasing tension on the tension members increases a diameter of the proximal opening.

One aspect of the disclosure is an intravascular blood pump, comprising: a collapsible blood conduit having an inner lumen for passing blood therethrough, wherein a proximal end of the blood conduit includes an impeller region configured to accommodate at least a portion of an impeller therein; and a plurality of struts and tension members configured to provide the impeller region resistance to deflection and to maintain a clearance for the impeller, the plurality of struts and tension members extending from the proximal end of the blood conduit and connected to a shaft, wherein: the plurality of struts forms a first conical structure having a large end pointing in a distal direction toward the blood conduit; and the tension members form a second conical structure having a large end pointing in the distal direction toward the blood conduit, wherein a portion of the second conical structure formed by the tension members is radially outside of the first conical structure formed by the struts.

The second conical structure may be formed by the tension members that extend further proximally along the shaft than the first conical structure formed by the struts. The plurality of struts may curve radially inward toward the shaft. The tension members may be substantially straight. The intravascular blood pump may further comprise a tension control element configured to control a degree of tension placed on the tension members, wherein higher tension on the tension members is associated with a higher stiffness of the impeller region. Proximal ends of the tension members may be connected to the tension control element. The tension control element may be configured to translate along the shaft. Proximal translation of the tension control element may increase tension on the tension members, and distal translation of the tension control element may decrease tension on the tension members. The plurality of struts and tension members may be connected to the shaft via one or more hubs. The one or more hubs may cover proximal ends of the plurality of struts and tension members. The one or more hubs may secure the proximal ends to the shaft.

One aspect of the disclosure is a method of using an intravascular blood pump, the method comprising: delivering the intravascular blood pump while in a collapsed state towards a heart; expanding a blood conduit of the intravascular blood pump in at least a portion of the heart, the expanded blood conduit defining an inner lumen, wherein a proximal end of the blood conduit includes an impeller region having at least a portion of an impeller therein; and causing the impeller to rotate and pump blood through the inner lumen of the blood conduit and out of the proximal end of the blood conduit, wherein the blood pump includes a plurality of struts and tension members configured to provide the impeller region resistance to deflection and to maintain a clearance for the impeller, the plurality of struts and tension members extending from the proximal end of the blood conduit and connected to a shaft, wherein: the plurality of struts forms a first conical structure having a large end pointing in a distal direction toward the blood conduit; and the tension members form a second conical structure having a large end pointing in the distal direction toward the blood conduit, wherein the second conical structure formed by the tension members is radially outside of the first conical structure formed by the struts.

The method may further comprise translating a tension control element along the shaft to control a degree of tension placed on the tension members, wherein higher tension on the tension members is associated with a higher stiffness of the impeller region. Proximal ends of the tension members may be connected to the tension control element. Translating the tension control element may comprise proximally translating the tension control element to increase tension on the tension members. Translating the tension control element may comprise distally translating the tension control element to decrease tension on the tension members.

One aspect of the disclosure is an intravascular blood pump, comprising: a collapsible blood conduit having an inner lumen for passing blood therethrough, a proximal end of the blood conduit including an impeller region configured to accommodate at least a portion of an impeller therein and including an opening to allow blood to pass therethrough; a plurality of struts and tension members extending from the proximal end of the blood conduit and connected to a shaft; and a tension control element configured to modulate a size of the opening by controlling a degree of tension placed on the tension members.

The tension control element may be configured to translate along the shaft. Proximal translation of the tension control element may increase tension on the tension members, and distal translation of the tension control element may decrease tension on the tension members. The plurality of struts may form a first conical structure having a large end pointing in a distal direction toward the blood conduit; and the tension members form a second conical structure having a large end pointing in the distal direction toward the blood conduit, wherein the second conical structure formed by the tension members is radially outside of the first conical structure formed by the struts. The second conical structure formed by the tension members may extend further proximally along the shaft than the first conical structure formed by the struts. The plurality of struts may curve radially inward toward the shaft. The tension members may be substantially straight. Proximal ends of the tension members may be connected to the tension control element. The tension control element may cover the proximal ends of the tension members to secure the tension members to the shaft.

One aspect of the disclosure is a method of using an intravascular blood pump, the method comprising: delivering the intravascular blood pump while in a collapsed state towards a heart; expanding a blood conduit of the intravascular blood pump in at least a portion of the heart, the expanded blood conduit defining an inner lumen, wherein a proximal end of the blood conduit including an impeller region configured to accommodate at least a portion of an impeller therein and including an opening to allow blood to pass therethrough, wherein the blood pump includes a plurality of struts and tension members extending from the proximal end of the blood conduit and connected to a shaft; causing the impeller to rotate and pump blood through the inner lumen of the blood conduit and out of the opening at the proximal end of the blood conduit; and activating a tension control element to modulate a size of the opening by controlling a degree of tension placed on the tension members.

Activating the tension control element may comprise translating the tension control element along the shaft. Translating the tension control element may comprise translating the tension control element proximally to increase tension on the tension members. Increasing tension on the tension members may increase a diameter of the opening. Increasing tension on the tension members may decrease a diameter of the opening. Translating the tension control element may comprise translating the tension control element distally to decrease tension on the tension members. Decreasing tension on the tension members may increase a diameter of the opening. Decreasing tension on the tension members may decrease a diameter of the opening.

One aspect of the disclosure is an intravascular blood pump, comprising: a collapsible blood conduit having an inner lumen for passing blood therethrough, wherein a proximal end of the blood conduit includes an impeller region configured to accommodate at least a portion of an impeller therein; and a plurality of struts and tension members configured to provide the impeller region resistance to deflection and to maintain a clearance for the impeller, the plurality of the struts and tension members extending from an impeller region of the blood conduit and connected to a proximal shaft, wherein each of the plurality of struts has a curved shape and each of the tension members has a straight shape.

Each of the plurality of struts may be joined to one or more tension members at or near a proximal end of the blood conduit, Adjacent struts may be radially equidistantly spaced apart from each other. Each of the plurality of struts may be connected with a first tension member and a second tension member at a junction region at a proximal end of the blood conduit. A span angle between the first tension member and the second tension member may be less than or equal to 75 degrees. A span angle between the first tension member and the second tension member may be less than or equal to 60 degrees. A tension member angle plus a strut angle may be less than 90 degrees, wherein the tension member angle is measured between a tension member line and a strut line, wherein the strut angle is measured between a strut line and a diameter line of the blood conduit, wherein the tension member line is measured from a point of connection of a tension member to the diameter line of the blood conduit to a point of connection of the tension member along a diameter line of proximal shaft, wherein the strut line is measured from a point of connection of a strut along the diameter line of blood conduit to a point of connection of the strut along the diameter line of the proximal shaft. The tension members may be straight. The tension members may be attached to a hub of the proximal shaft. The hub may house a bearing assembly therein.

These and other details and aspects are described herein.

### BRIEF DESCRIPTION OF THE DRAWINGS

FIG. 1 is a side view of an exemplary blood pump that includes an expandable scaffold that supports a blood conduit with an impeller housed therein.

FIG. 2 is a side view of an impeller region of an exemplary blood pump experiencing a lateral load.

FIGS. 3A and 3B are side views of an impeller region of another exemplary blood pump having tensioning members: FIG. 3A shows the blood pump experiencing the same lateral load as the blood pump of FIG. 2; and FIG. 3B shows the blood pump experiencing a lateral load that is 15 times the lateral load of the blood pump in FIG. 2.

FIG. 4 is a graph comparing stiffnesses of the blood pump of FIG. 2 and the blood pump of FIGS. 3A-3B.

FIG. 5 shows a side view of an impeller region of an exemplary blood pump having tension members and a tension control element.

FIG. 6A-6C show side views of the impeller region of the blood pump of FIG. 5 with different levels of tension applied to the tension members.

FIG. 7 is a graph showing stiffness as a function of preloading of the tension members for the blood pump of FIGS. 5 and 6A-6C.

FIGS. 8A illustrates exemplary dimensions defining aspects of the tension members of the blood pumps of FIGS. 3A-3B or FIGS. 5-7.

FIGS. 8B illustrates additional exemplary dimensions defining aspects of the tension members of the blood pumps of FIGS. 3A-3B or FIGS. 5-7.

### DETAILED DESCRIPTION

The present disclosure is related to medical devices, systems, and methods of use and manufacture. Medical devices herein may include a distal pump portion (which may also be referred to herein as a working portion) adapted to be disposed within a physiologic vessel, wherein the distal pump portion includes one or more components that act upon fluid. For example, pump portions herein may include one or more rotating members that when rotated, can facilitate the movement of a fluid such as blood.

Any of the disclosure herein relating to an aspect of a system, device, or method of use can be incorporated with any other suitable disclosure herein. For example, a figure describing only one aspect of a device or method can be included with other embodiments even if that is not specifically stated in a description of one or both parts of the disclosure. It is thus understood that combinations of different portions of this disclosure are included herein.

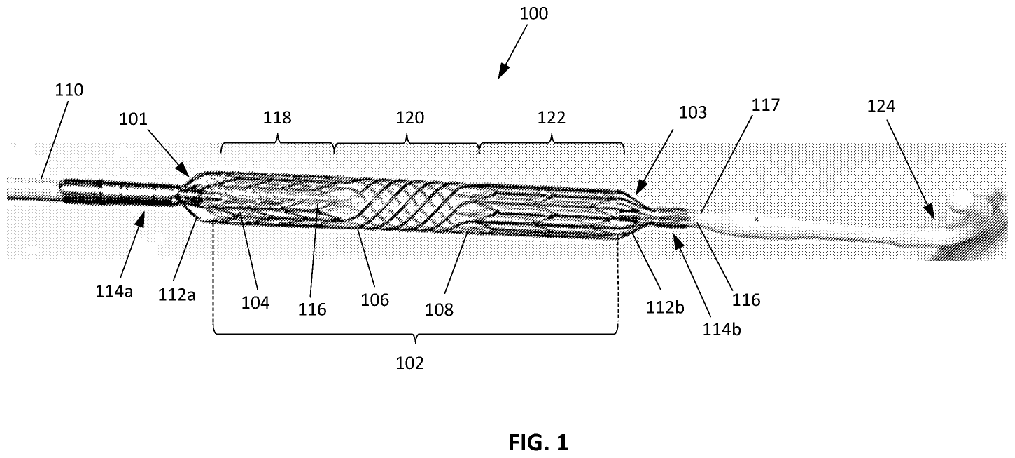

FIG. 1 shows a side view of an exemplary intravascular blood pump 100. The blood pump 100 includes an expandable/collapsible blood conduit 102 that is configured to transition between an expanded state, as shown in FIG. 1, and a collapsed state (not shown). For example, the blood conduit 102 may be in the collapsed state when confined within a delivery catheter for delivery to the heart, expanded upon release from the delivery catheter for blood pumping, and collapsed back down within the delivery catheter (or other catheter) for removal from heart. When in the expanded state, the blood conduit 102 is radially expanded so as to form an inner lumen for passing blood therethrough. When in the expanded state, the inner lumen of the blood conduit 102 may be configured to house one or more impellers therein.

The blood conduit 102 includes a first (e.g., proximal) opening 101 and a second (e.g., distal) opening 103. In some examples, the first opening 101 and second opening 103 may be configured as an inlet and outlet for blood. For example, blood may largely enter the blood conduit 102 via the second (e.g., distal) opening 103 and exit the blood conduit 102 via the first (e.g., proximal) opening 101. In such cases, the second opening 103 acts as a blood inlet and the first opening 101 acts as a blood outlet. The one or more impellers (e.g., impeller 104) may be configured to pump blood from the inlet toward the outlet.

The blood conduit 102 includes a tubular expandable/collapsible scaffold 106 that provides structural support for a membrane 108 that covers at least a portion of inner surfaces and/or outer surfaces of the scaffold 106. The scaffold 106 includes a material that forms a pattern of axially and radially extending elements that define a pattern of openings. The membrane 108 may cover the openings to retain the blood within the lumen of the blood conduit 102. The scaffold 106 may be unitary and may be made of a single piece of material. For example, the scaffold 106 may be formed by cutting (e.g., laser cutting) a tubular shaped material. Exemplary materials for the scaffold 106 may include one or more of: nitinol, cobalt alloys, and polymers, although other materials may be used.

The blood pump 100 includes proximal struts 112a that extend from the scaffold 106 near the first opening 101 (e.g., blood outlet region) and distal struts 112b that extend from the scaffold 106 near the second opening 103 (e.g., blood inlet region). In this example, the proximal ends of the proximal struts 112a are coupled to a shaft 110 (e.g., proximal shaft) at a first hub 114a. The first hub 114a may be part of the shaft 110 or be coupled to the shaft 110. At least a portion of the first hub 114a may be configured to cover the proximal ends of the proximal struts 112a. For example, the first hub 114a may have an annular shape that covers the proximal ends of the proximal struts 112a, which may secure (or help secure) the proximal struts 112a to the shaft 110. The distal ends of the distal struts 112b are coupled to shaft 117 (e.g., distal shaft) at a second hub 114b. The second hub 114b may be part of the shaft 117 or be coupled to the shaft 117. At least a portion of the second hub 114b may be configured to cover the distal ends of the distal struts 112b. For example, the second hub 114b may have an annular shape that covers the distal ends of the distal struts 112b, which may secure (or help secure) the distal struts 112b to the shaft 117. In this example, the first hub 114a and/or the second hub 114a includes a bearing assembly housed therein and through which a central drive cable 116 extends. The drive cable 116 may be operationally coupled to and configured to rotate the impeller 104 (and/or one or more additional impellers, if present).

In this example, the blood pump 100 includes an impeller 104 within a proximal portion of at least a portion of the blood conduit 102. At least a portion of the impeller 104 may extend proximally past the blood conduit 102 and the scaffold 106, and into an outlet region of the pump 100. For example, at least a portion of the impeller 104 may be axially aligned with at least a portion of the proximal struts 112a (and with at least a portion of tension members as described herein).

In some cases, the blood pump 100 may include more than one impeller. For example, the blood pump 100 may include a first impeller (e.g., impeller 104) a second impeller in a distal region 122 of the fluid conduit 102. In some cases, the blood pump 100 may include more than two impellers.

In some cases, the shaft 110 (e.g., proximal shaft) may be continuously connected to the shaft 117 (e.g., distal shaft). For example, the shafts 110 and 117 may form a continuous shaft through the blood conduit 102 with the drive cable 116 housed therein. In other examples, the shaft 110 (e.g., proximal shaft) may be separate from the shaft 117 (e.g., distal shaft). For example, the drive cable 116 may extend distally from the shaft 110 (e.g., proximal shaft) to the shaft 117 (e.g., distal shaft), where the drive cable 116 is not housed within the shafts 110 and 117 in the region within the blood conduit 102.

In some cases, the impeller 104 is fully positioned axially within the blood conduit 102. In other cases, a proximal portion of the impeller 104 may be positioned at least partially outside of the blood conduit 102. For example, at least a portion of the impeller 104 may be positioned axially distal to the proximal end of the blood conduit 102. In some cases, a distal portion (e.g., distal end) of the impeller 104 may be in axial alignment with a distal portion of the struts 112a.

The blood conduit 102 and the scaffold 106 may be characterized as having a proximal region 118, a central region 120, and a distal region 122. The central region 120 may be configured to be placed across a valve (e.g., aortic valve) such that the proximal region 118 is at least partially within a first heart region (e.g., ascending aorta) and the distal region 122 is at least partially within a second heart region (e.g., left ventricle). The proximal region 118 (and in some cases the distal region 122) may be configured to house at least a portion of an impeller therein. The proximal region 118 may (and in some cases the distal region 122) have a stiffness sufficient to withstand deformation during operation of the blood pump 100 when within the beating heart and to maintain clearance (i.e., a gap) between an impeller region of the blood pump 100 and the rotating impeller 104. The impeller region may be configured to accommodate at least a portion of one or more impellers therein. The distal region 122 includes the second (e.g., distal) opening 103 of the blood conduit 102, and may serve as the blood inlet for the blood conduit 102.

In some examples, the central region 120 may be less rigid relative to the proximal region 118 (and in some cases the distal region 122). The higher flexibility of the central region 102 may allow the central region 102 to deflect when a lateral force is applied on a side of the blood conduit 102, for example, as the blood conduit 102 traverses through the patient's blood vessels and/or within the heart. For example, the central region 120 may be configured to laterally bend upon a lateral force applied to the distal region 122 and/or the proximal region 118. In some cases, it may be desirable for the central region 120 to laterally bend as the blood conduit 102 traverses the ascending aorta and to temporarily assume a bent configuration when the blood conduit 102 is positioned across an aortic valve. In this example, the central region 120 includes a helical arrangement of longitudinally running elongate elements configured to provide flexibility for lateral bending. In some examples, a distal tip 124 of the blood pump 100 includes a curved to form an atraumatic tip.

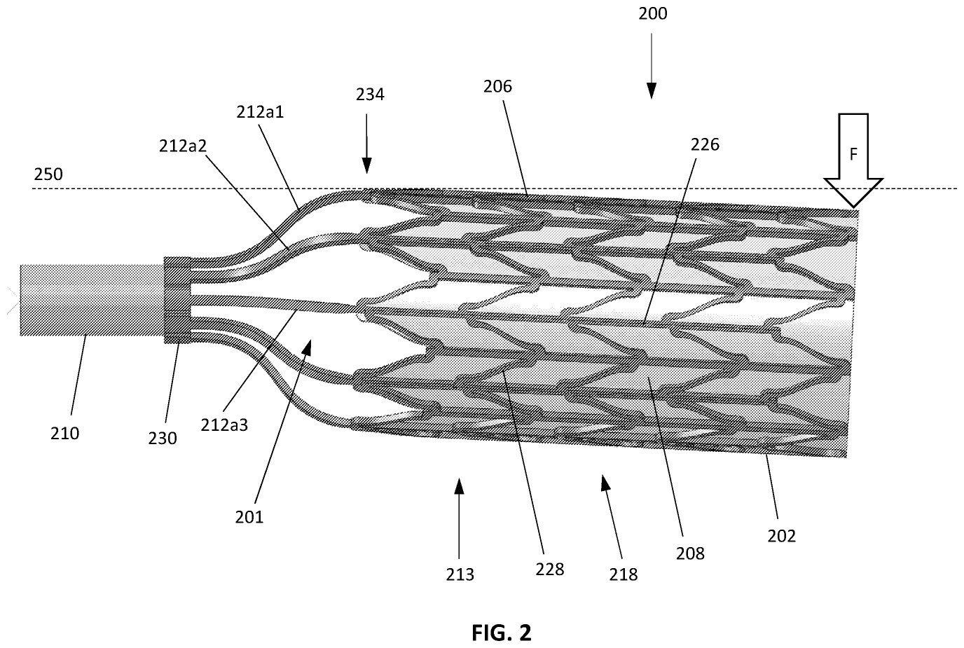

FIG. 2 shows a close up view of an impeller region 213 of an exemplary blood pump 200 (similar to the blood pump 100) in an expanded state. As with the blood pump 100 described above, a blood conduit 202 can include a scaffold 206 and a membrane 208 that covers at least a portion of inner surfaces and/or outer surfaces of the scaffold 206 to retain the blood within the blood conduit 202. The scaffold 206 may be configured to maintain a shape of the scaffold 206 in the expanded state and radially contract to a collapsed state when a radially compressive force is placed on the scaffold 206 (e.g., when collapsed within a delivery catheter). The scaffold 206 includes a pattern of axially and radially extending elements that define a pattern of openings, which may be covered by the membrane 208.

In the example of FIG. 2, the scaffold 206 includes a series of axially extending elongate elements 226 that extend from the proximal region 218, through the central region (e.g., 120 in FIG. 1), and the distal region (e.g., 122 in FIG. 1). In the proximal region 218, the axially extending elongate elements 226 are connected by connector elements 228 (which extend radially between the elongate elements 226) to provide structural rigidity to the blood conduit 202 so that the blood conduit 202 can withstand deformation (e.g., deflection), for example, from forces placed on the blood conduit 202 by tissue (e.g., walls) of the heart. The impeller region 213 may correspond to an axial region of the blood pump 200 where the impeller 214 resides. The impeller region 213 may include at least a portion of the proximal region 218 of the blood conduit 202/scaffold 206. In some cases, the impeller region 213 may include at least a portion of the proximal struts (e.g., 212a1, 212a2 and 212a3). At least a portion of an impeller may be axially aligned with a proximal portion of the blood conduit 102 and/or with at least a portion of the proximal struts (e.g., 212a1, 212a2 and 212a3). That is, in some cases, at least a portion of the impeller may extend proximally past a proximal end of the blood conduit 206 (e.g., scaffold 206 and/or membrane 208). As described above, the impeller region 213 may be sufficiently rigid to allow clearance between the impeller and the impeller region 213 of the blood pump 200 to be maintained during operation of the blood pump 100 (e.g., within the beating heart).

The struts (e.g., 212a1, 212a2 and 212a3) may extend from the proximal end of the blood conduit 202 (e.g., from the scaffold 206) and connect to a proximal shaft 210. The struts (e.g., 212a1, 212a2 and 212a3) may be arranged to provide space for an opening 201 for blood to flow therethrough (e.g., outlet). In some cases, the struts (e.g., 212a1, 212a2 and 212a3) may be directly connected to the shaft 210. For example, the struts (e.g., 212a1, 212a2 and 212a3) may be welded and/or adhered (e.g., via adhesive) to the shaft 210. In some cases, a hub (e.g., hub 114a in FIG. 1) may cover proximal ends 230 (also referred to as feet) of the struts (e.g., 212a1, 212a2 and 212a3) to help secure the struts (e.g., 212a1, 212a2 and 212a3) to the shaft 210. In some cases, the struts (e.g., 212a1, 212a2 and 212a3) may be indirectly connected to the shaft 210. For example, the proximal ends 230 of the struts (e.g., 212a1, 212a2 and 212a3) may be coupled to one or more connectors (e.g., ring, collar) that is coupled the shaft 210.

The struts (e.g., 212a1, 212a2 and 212a3) may be connected to the blood conduit 100 (e.g., to the scaffold 206) at a junction region 234. In the example shown, the junction region 234 is at a proximal end of the blood conduit 202 and/or scaffold 206. In other examples, the junction region 234 may be at a more axially distal location of the blood conduit 202 and/or scaffold 206.

In some examples, the struts (e.g., 212a1, 212a2 and 212a3) may cooperate to form a conical structure, with a large end of the conical structure pointing in a distal direction toward the blood conduit 302. The struts (e.g., 212a1, 212a2 and 212a3) may be bent radially inward as they approach the proximal shaft 210. In this example, the struts (e.g., 212a1, 212a2 and 212a3) are curved radially inward toward the proximal shaft 210. In some cases, the struts (e.g., 212a1 and 212a2) may be axially continuous with the axially extending elongate elements 226 of the scaffold 206. In some examples, the struts (e.g., 212a1, 212a2 and 212a3) may cooperate to form a non-conical three-dimensional shape, such as a prism, tetrahedron, spherical, ovoid, cuboid, or cylinder shape.

In the example of FIG. 2, the struts (e.g., 312a1, 312a2 or 312a3) are connected to the shaft at the same axial position along the shaft 210. In other examples, one or more of the struts (e.g., 312a1, 312a2 or 312a3) may be connected the shaft 210 at different axial positions along the shaft 210.

Adjacent struts (e.g., 212a1, 212a2 and 212a3) may be radially equidistantly spaced apart from each other, such as shown in the example of FIG. 2. In other examples, adjacent struts (e.g., 212a1, 212a2 and 212a3) may be radially spaced apart by non-equal distances. For example, a first strut 212a1 may be radially spaced apart from an adjacent second strut 212a2 by a first distance, and the second strut 212a2 may be radially spaced apart from an adjacent third strut 212a3 by a second distance that is different than the first distance.

In some cases, the struts (e.g., 212a1, 212a2 and 212a3) may be shape set so as to radially expand the scaffold 206 upon release from a delivery catheter. When the blood pumping procedure is complete, the struts (e.g., 212a1, 212a2 and 212a3) may be collapsed radially inward upon retraction within the delivery catheter (or other catheter). The proximal ends 230 (also referred to as feet) of the struts (e.g., 212a1, 212a2 and 212a3) may be shaped and sized to facilitate connection to the proximal shaft 210. For example, the proximal ends 230 may be wider than other portions of the struts (e.g., 212a1, 212a2 and 212a3) for engagement with a retaining component to secure the proximal ends 230 to the proximal shaft 210.

In this example, the blood pump 200 includes ten proximal struts (e.g., 212a1, 212a2 and 212a3); however, the blood pumps described herein may include any number of proximal struts (e.g., 1, 2, 3, 4, 5, 6, 7, 8, 9, 10, 11, 12, 13, 14, or more.). In some cases, the material of the struts may be made of the same material as the scaffold (e.g., nitinol, cobalt alloy, and/or polymer).

Although the struts (e.g., 212a1, 212a2 and 212a3) provide some resistance to deflection, a lateral force “F” applied to the scaffold 206 may result in some lateral deflection with respect to a horizontal reference line 250. Such lateral force “F” may be from tissue (e.g., walls) of the heart as the heart is beating. To address this, the proximal end of the blood conduit 202 may include additional structural supports, such as tension members, as described herein.

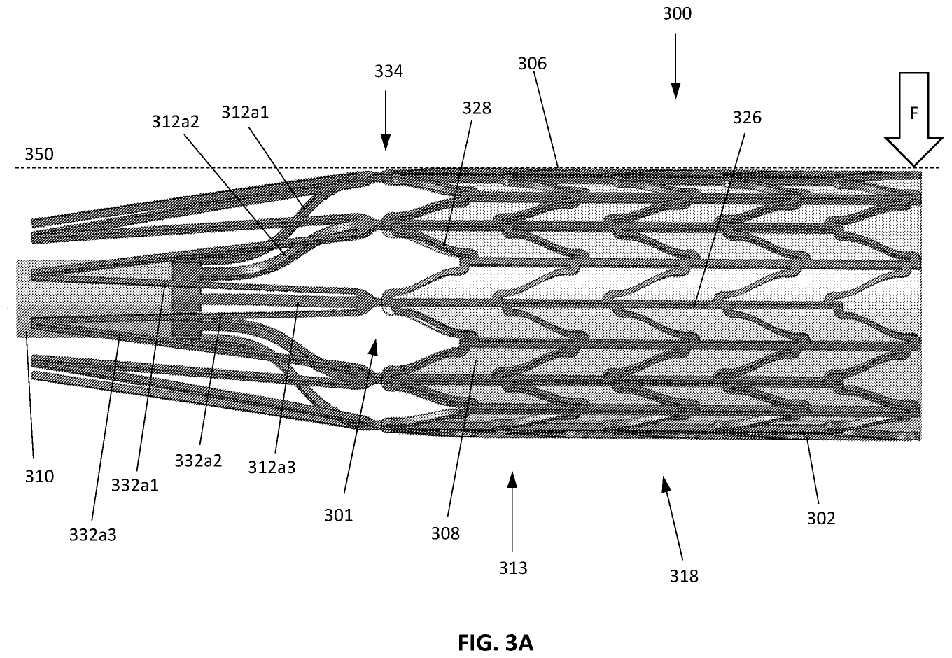

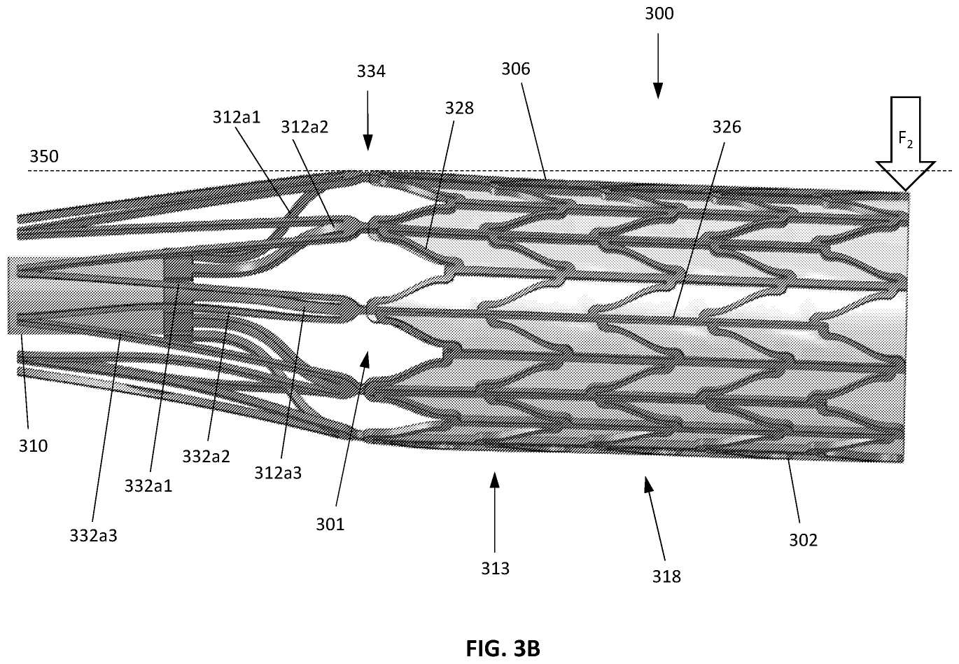

FIGS. 3A and 3B show close up views of an impeller region 313 of another exemplary blood pump 300 having similar features as the blood pump 200 of FIG. 2 but with the addition of tension members (e.g., 332a1, 332a2 and 332a3) (also referred to herein as stiffening members). The addition of tension members (e.g., 332a1, 332a2 and 332a3) may provide extra stiffness to the proximal end of the scaffold 306, thereby making the struts (e.g., 312a1, 312a2 and 312a3) and proximal region 318 of the blood conduit 302 more resistant to deflection, and making the impeller region 213 of the blood conduit 302 more capable of maintaining clearance between the impeller region 313 and an impeller housed therein. That is, the addition of the tension members (e.g., 332a1, 332a2 and 332a3) may increase the stiffness (e.g., lateral stiffness) of the impeller region 313 compared to the impeller region 213 of blood pump 200. For example, the addition of the tension members (e.g., 332a1, 332a2 and 332a3) may result in significantly less deflection with respect to a horizontal reference line 350 when the same amount of lateral force “F” is applied to the scaffold 306 compared to the blood pump 200. The increased resistance to deformation may also help to keep the opening 301 (e.g., for the outflow of blood) of the blood conduit 302 fully opened, thereby increasing the efficiency of the blood pump 300. The blood pumps described herein may include any number of tension members (e.g., 1, 2, 3, 4, 6, 8, 10, 12, 14, 16, 18, 20, 24, 30, or more.). In this example, the blood pump 300 includes 20 tension members (e.g., two times the number of struts).

Like struts (e.g., 312a1, 312a2 and 312a3), the tension members (e.g., 332a1, 332a2 and 332a3) are elongate elements that extend from the blood conduit 302 in a proximal direction and radially inward toward a proximal shaft 310. The struts (e.g., 312a1, 312a2 and 312a3) may be proximally coupled to the shaft 310 at a first axial region along the shaft 310, and the tension members (e.g., 332a1, 332a2 and 332a3) may be proximally coupled to the shaft 310 at a second axial region along the shaft 310. The first axial region along the shaft 310 may be different than the second axial region along the shaft 310. In this example, the second axial region extends further proximally along the shaft 310 relative to the first axial region. That is, the tension members (e.g., 332a1, 332a2 and 332a3) extend further proximally along the proximal shaft 310 than the struts (e.g., 312a1, 312a2 and 312a3). This may allow the tension members (e.g., 332a1, 332a2 and 332a3) to provide more tension to stiffen the impeller region 313 compared to the struts (e.g., 312a1, 312a2 and 312a3). However, in other examples, the first axial region may be the same axial position as the second axial region along the shaft 310, or may extend further proximally along the shaft 310 relative to the second axial region. This may still allow the tension members (e.g., 332a1, 332a2 and 332a3) to provide more tension to stiffen the impeller region 313 compared to there being no tension members (e.g., 332a1, 332a2 and 332a3).

In some cases, the tension members (e.g., 332a1, 332a2 and 332a3) may be directly connected to the shaft 310. For example, the tension members (e.g., 332a1, 332a2 and 332a3) may be welded and/or adhered (e.g., via adhesive) to the shaft 310. In some cases, a hub (e.g., hub 114a in FIG. 1) may cover proximal ends of the tension members (e.g., 332a1, 332a2 and 332a3) to help secure the tension members (e.g., 332a1, 332 a2 and 332a3) to the shaft 310. In some cases, the tension members (e.g., 332a1, 332a2 and 332a3) may be indirectly connected to the shaft 310. For example, the proximal ends of the tension members (e.g., 332a1, 332a2 and 332a3) may be coupled to a connector (e.g., ring, collar) that is coupled the shaft 310.

In the example of FIGS. 3A and 3B, at least a portion of the tension members (e.g., 332a1, 332a2 and 332a3) are radially outside of the struts (e.g., 312a1, 312a2 and 312a3). In other examples, at least a portion of the tension members (e.g., 332a1, 332a2 and 332a3) may be radially inside of the struts (e.g., 312a1, 312a2 and 312a3).

The tension members 332 together may form a second conical structure having a large end pointing in a distal direction toward the blood conduit and that is radially outside of the first conical structure formed by the struts (e.g., 312a1, 312a2 and 312a3). As discussed above, in this example, the tension members (e.g., 332a1, 332a2 and 332a3) extend further proximally along the proximal shaft 310 than the struts (e.g., 312a1, 312a2 and 312a3). Thus, in this example, the second conical structure formed by the tension members (e.g., 332a1, 332a2 and 332a3) extends further proximally along the shaft 310 than the first conical structure formed by the struts (e.g., 312a1, 312a2 and 312a3). In other examples, the first conical structure formed by the struts (e.g., 312a1, 312a2 and 312a3) may extend by the same extent along the shaft 310 as the second conical structure formed by the tension members (e.g., 332a1, 332a2 and 332a3), or may extend further proximally along the proximal shaft 310 than the first conical structure formed by the tension members (e.g., 332a1, 332a2 and 332a3).

In this example, the tension members 332 are substantially straight in shape (e.g., as opposed to curved like the struts 312a1, 312a2 and 312a3). However, the tension members (e.g., 332a1, 332a2 and 332a3) may have any shape to provide a different degree of tension on the impeller region 313. For example, the tension members (e.g., 332a1, 332a2 and 332a3) may curve radially inward toward the shaft 310 (e.g., similar to the struts 312a1, 312a2 and 312a3) to provide a different degree of tension on the impeller region 313.

The tension members (e.g., 332a1, 332a2 and 332a3) may be connected to the blood conduit 302 in any of a number of ways. In the example shown, the tension members (e.g., 332a1, 332a2 and 332a3) and struts (e.g., 312a1, 312a2 and 312a3) are connected to a proximal end of the scaffold 306, specifically to proximal ends of a portion of the axially extending elongate elements (e.g., 326) of the scaffold 306 at a junction region 334. In other examples, the junction region 334 for the tension members (e.g., 332a1, 332a2 and 332a3) and/or the struts (e.g., 312a1, 312a2 and 312a3) may be at a more axially distal location of the blood conduit 302 and/or scaffold 306.

In the example of FIGS. 3A and 3B, the tension members (e.g., 332a1, 332a2 and 332a3) and the struts (e.g., 312a1, 312a2 and 312a3) are coupled to each other at or near the junction region 334 where they connect to the scaffold 306. For example, two tension members 332a1 and 332a2 converge and connect to a strut 312a3 at the junction region 334. That is, at or near the junction region 334, each strut (e.g., 312a1, 312a2 and 312a3) is adjacent to a tension member (e.g., 332a1, 332a2 and 332a3) on either side. This configuration may provide balanced stiffening support for the proximal end of the scaffold 306 while providing space for the opening 301 (e.g., for the outflow of blood). Although the example shown includes two tension members (e.g., 332a1, 332a2 and 332a3) for each of the struts (e.g., 312a1, 312a2 and 312a3), the blood pumps described herein may include any number of tension members per struts (e.g., 1, 2, 3, 4, 5, 6, 7, or more.) and may include any total number of tension members (e.g., 1, 2, 3, 4, 5, 6, 7, 8, 9, 10, 11, 12, 13, 14, or more.). In addition, the attachment locations of the distal ends of the tension members (e.g., 332a1, 332a2 and 332a3) are not necessarily limited to being at or near the junction region 334. For example, the distal ends of tension members (e.g., 332a1, 332a2 and 332a3) may be attached to portions of the struts (e.g., 312a1, 312a2 and 312a3) proximal to the junction region 334 and/or may be attached to the scaffold 306 distal to the junction region 334. In some cases, the struts (e.g., 312a1, 312a2 and 312a3) do not connect with the tension members (e.g., 332a1, 332a2 and 332a3).

In the example in FIGS. 3A and 3B, proximal ends of each tension member (e.g., 332a1, 332a2 and 332a3) connects at the proximal shaft 310 adjacent to or abutting a tension member (e.g., 332a1, 332a2 and 332a3) belonging to the adjacent strut (e.g., 312a1, 312a2 and 312a3). For example, tension member 332a2 converges with tension member 332a3 as they run proximally toward and connect to the proximal shaft 310. The adjacent tension members (e.g., 332a1, 332a2 and 332a3) may be connected together at the proximal shaft 310, may be connected near each other at the proximal shaft 310, or may be separate from each other at the proximal shaft 310. In the example of FIGS. 3A and 3B, the tension members (e.g., 332a1, 332a2 and 332a3) are connected to the shaft at the same axial position (e.g., second axial region) along the shaft 310, and the struts (e.g., 312a1, 312a2 or 312a3) are connected to the shaft at the same axial position (e.g., first axial region) along the shaft 310. In other examples, one or more of the tension members (e.g., 332a1, 332a2 and 332a3) may be connected the shaft 310 at different axial positions along the shaft 310, and/or the struts (e.g., 312a1, 312a2 or 312a3) may be connected the shaft 310 at different axial positions along the shaft 310.

Adjacent tension members (e.g., 332a1, 332a2 and 332a3) may be spaced apart from each other in any way. In the example of FIGS. 3A and 3B, the tension members (e.g., 332a1, 332a2 and 332a3) are arranged in pairs that are radially equidistantly spaced apart from each other. In other examples, the tension members (or tension member pairs) may be radially spaced apart by non-equal distances.

The tension members (e.g., 332a1, 332a2 and 332a3) may be made of the same or different material as the struts (e.g., 312a1, 312a2 and 312a3) and/or scaffold 306. In some examples, the tension members (e.g., 332a1, 332a2 and 332a3) (and/or struts (e.g., 312a1, 312a2 and 312a3)) may be formed by cutting (e.g., laser cutting) the tubular shaped material forming the scaffold 306. In other examples, the tension members (e.g., 332a1, 332a2 and 332a3) (and/or struts (e.g., 312a1, 312a2 and 312a3)) may be wires or cables that are attached to the scaffold 306. Exemplary materials for the tension members (e.g., 332a1, 332a2 and 332a3) (and/or struts (e.g., 312a1, 312a2 and 312a3)) may include one or more of: nitinol, stainless steels, cobalt-chrome alloys, and polymers, although other materials may be used.

FIG. 3B shows the blood pump 300 experiencing a lateral load “F₂” that is 15 times greater than the lateral load “F” in order to achieve a similar degree of deflection as the blood pump 200 with respect to the horizontal reference line 350.

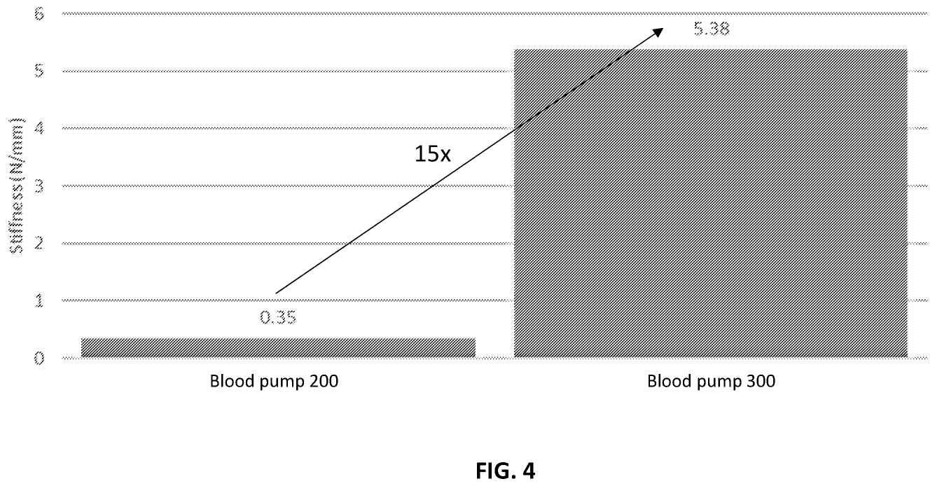

FIG. 4 is a graph showing results of finite element analysis comparing the stiffnesses (in Newtons per millimeter) of the proximal regions of the blood pump 200 of FIG. 2 and blood pump 300 of FIGS. 3A-3B. These results indicate that the addition of tension members 332 increases the stiffness 15 times compared to the blood pump without the tension members 332.

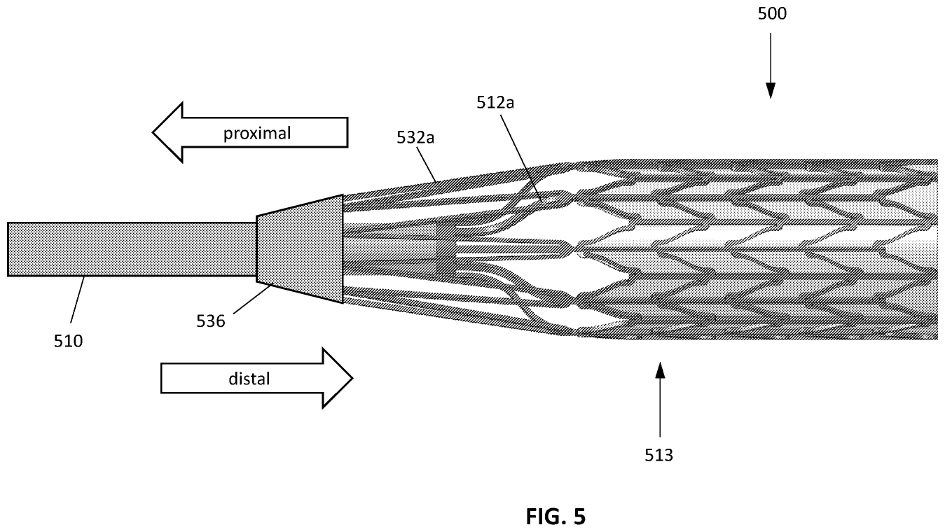

FIG. 5 shows a close up up view of an impeller region 513 of further exemplary blood pump 500 having similar features as the blood pump 300, where the tension members 532a are coupled to a shaft 510 (e.g., proximal shaft) by a second hub 536. The second hub 536 may have an annular shape that encircles the shaft 510. In this example, the second hub 536 is axially tapered, with a distal diameter greater than a proximal diameter to match the taper of the tension members 532a. In some examples, the second hub 536 may be axially fixed relative to the shaft 510. In other examples, the second hub 536 may be axially movable relative to the shaft 510. In cases where the second hub 536 is movable, moving the second hub 536 axially may change the amount of tension placed on the tension members 532a. In these cases, the second hub 536 may be referred to as a tension control element. That is, the tension control element 536 may be operationally coupled to the tension members 532a and configured to control a degree of tension placed on the tension members 532a. Increasing tension on the tension member 532a may increase the bend resistance of the struts 512a and increase the bending stiffness of the impeller region 513. In this way, increasing tension on the tension members 532a may further help to maintain clearance between the impeller and the impeller region 513 during operation of the blood pump 500. The tension control element 536 may be used to reduce the stiffness, for example, before, during and/or after sheathing of the blood pump 500 within a catheter (e.g., delivery catheter), and to increase the stiffness, for example, during or after expansion and deployment of the blood pump 500 from a catheter (e.g., delivery catheter).

In some examples, the tension control element 536 is configured to translate axially in a proximal direction to increase tension on the tension members 532a, and in a distal direction to decrease tension on the tension members 532a. The tension control element 536 may be activated (e.g., translated) using any of a number of mechanisms. In some cases, one or more wires or cables are attached to the tension control element 536 and extend proximally to a handle. The wire(s)/cable(s) may be housed within the proximal shaft 510 and/or be positioned outside of the proximal shaft 510. The user may pull and/or release tension on the one or more wires or cables at the handle. In some cases, the user may actuate a button and/or lever on the handle to activate/release the tension control element 536. In some cases, the tension control element 536 is automatically activated/released.

In some cases, the tension control element 536 is configured to be locked or unlocked. For example, a locking feature(s) may be configured to engage the cable(s)/wire(s) to place tension on and maintain the tension control element 536 at an axial position along the shaft 510. The locking feature(s) may be configured to release tension on the cable(s)/wire(s) and to allow the tension control element 536 to translate distally to a more axially distal position. In some cases, the locking feature(s) may include an indexing feature that is configured to lock the tension control element 536 at one or more predetermined axial positions along the shaft 510. The locking feature(s) may be located in/on the handle and/or in/on the tension control element 536. The locking feature(s) may be configured to be actuated manually or automatically. In some examples, a separate actuation element is configured to lock or unlock the tension control element 536. In one example, the locking feature(s) is/are be used to maintain tension on the tension members 532a in a locked state during and/or after deployment of the blood pump 500 from a catheter (e.g., delivery catheter), and to release tension on the tension members 532a in an unlocked state before, during and/or after sheathing of the blood pump 500 within a catheter (e.g., delivery catheter).

The tension control element 536 may be axially located in any of a number of locations along the proximal shaft 510. In some examples, the tension control element 536 may configured to translate over at least a portion of a hub (e.g., 114a in FIG. 1) that covers proximal ends of the struts 512a. As described above, in some cases, the hub (e.g., 114a) may include a bearing assembly housed therein.

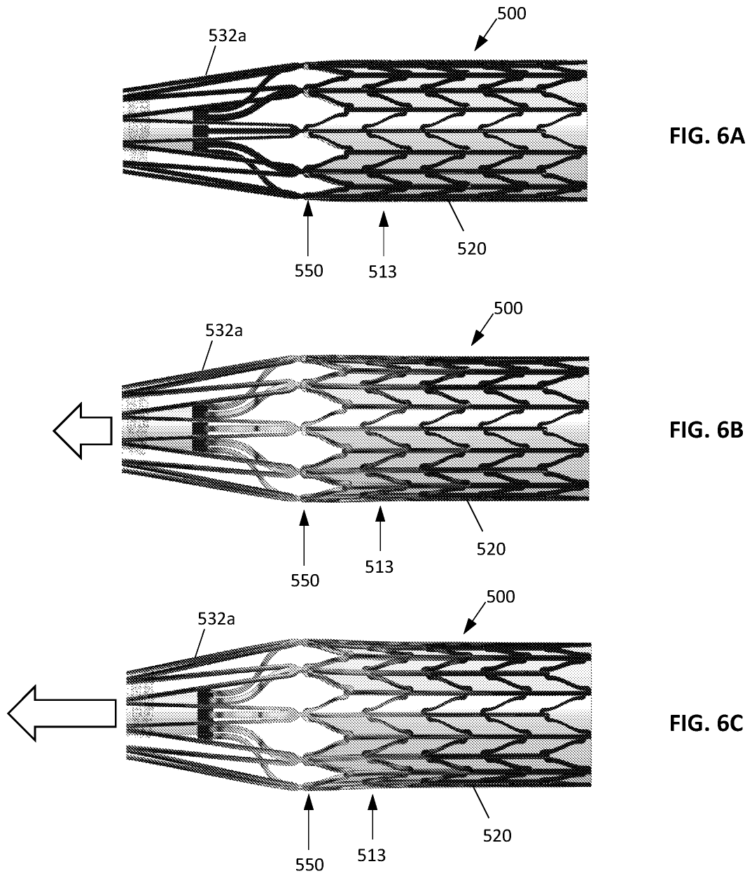

In some cases, the tension members may be used to modify a shape of the inlet and/or outlet opening of the blood conduit. To illustrate, FIGS. 6A-6C show the impeller region 513 of the blood pump 500 with different levels of tension applied to tension members 532a such as with a tension control element similar to described above. FIG. 6A shows the impeller region 513 when no tension is applied to the tension members 532a. A proximal opening 550 of the blood conduit 502 has a constant (unchanged) first diameter since no tension is applied to the tension members 532a. FIG. 6B shows the blood pump 500 when a first amount of force is applied in the proximal direction on the tension members 532a (e.g., by translating a tension control element 536). The first applied tension causes the diameter of the opening 550 to increase (e.g., flare) to a second diameter. FIG. 6C shows the blood pump 500 when a second amount of force, greater than the first amount of force, is applied in the proximal direction on the tension members 532a (e.g., by further translating the tension control element 536). The second applied tension causes the diameter of the opening 550 to increase (e.g., flare) to a third diameter that is greater than the second diameter. In this way, the tension members 532a may be preloaded by a specified amount to increase or decrease the diameter 550 of the opening 550. This aspect may be useful, for example, to modulate the flow of fluid (e.g., blood) through the opening 550 (e.g., outlet), thereby modulating a performance of the blood pump 500. This post-deployment modulation of the geometry of the opening 550 avoids potential downsides of shape-setting a flared geometry of the opening 550 into a resting state of the scaffold (e.g., which may be at the expense of additional sheathing strains and forces).

In some examples, the blood pump 500 may include more than one tension control element 536. For example, a first set of tension members 532a may be connected to a first tension control element and a second set of tension members 532a may be connected to a second tension control element. The first and second tension control elements may be at different axial positions along the shaft 510. The first and second tension control elements may be configured to translate axially independently with respect to each other, thereby allowing for independent choosing of tension placed on the first and second sets of tension members 532a. In other cases, the first and second tension control elements may be configured to translate axially in tandem.

In some cases, the tension control element 536 may be adjusted and locked to an axial position prior to delivery of the blood pump 500 into the subject's heart. For example, the tension control element 536 may be adjusted to a position to impart a predetermined amount of tension on the 513 prior to delivering the blood pump 500 into the heart. In some cases, the tension control element 536 may be adjusted while the blood pump 500 is in the subject's heart. For example, performance of the blood pump 500 may be monitored while in the heart, and the tension control element 536 may be adjusted (e.g., using a proximal handle of the blood pump 500) to improve the performance of the blood pump 500 while in the heart. Types of performance may include the pumping efficiency (e.g., as monitored by one or more sensors of the blood pump 500), amount of deflection of the blood pump 500 (e.g., as monitored by imaging) and/or location of the blood pump 500 relative to structures of the heart (e.g., as monitored by imaging).

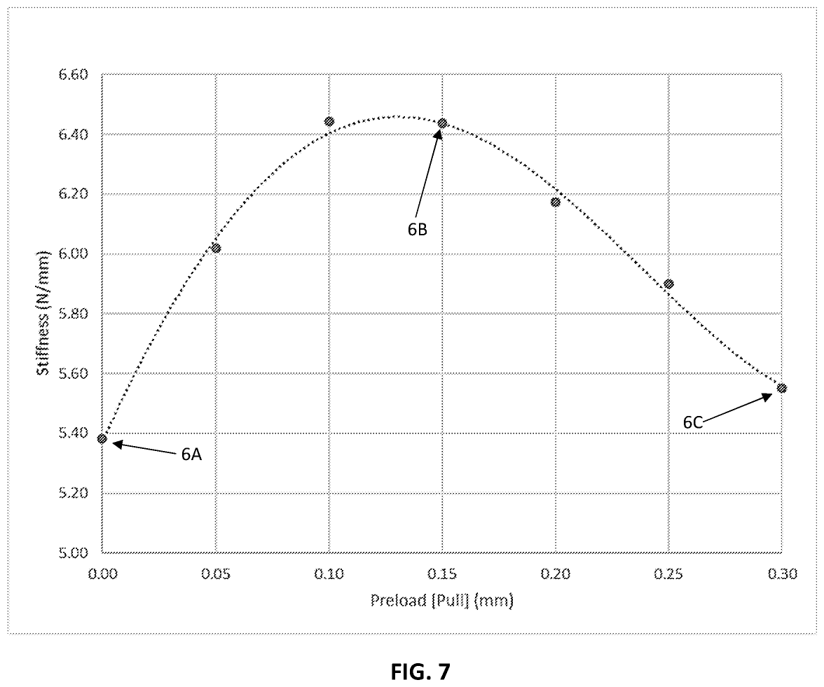

FIG. 7 is a graph showing finite analysis results of stiffness (N/mm) of the impeller region 513 as a function of preloading (as measured by distance (mm) of proximal translation of the tension control element 536), including the preload levels of tension shown in FIGS. 6A-6B. These results show that the stiffness of the impeller region 513 may increase with increasing tension on the tensioning members 532a until a maximum stiffness is achieved, after which the stiffness may decrease with increasing tension on the tensioning members 532a. Thus, the outlet opening modulation may be weighed against stiffness when translating the tension control element 536. That is, there may be an ideal range of preload that optimizes for tension. In this example, such optimal preload ranges from about 0.05 mm to about 0.25 mm of pull.

Referring to FIGS. 7 and 6A, no preload applied to the tension members 532a results in an impeller region stiffness of approximately 5.4 N/mm. However, referring to FIGS. 7 and 6B, a preload or pull of 0.15 mm is applied to the tension members 532a results in an increased impeller region stiffness of approximately 6.4 N/mm.

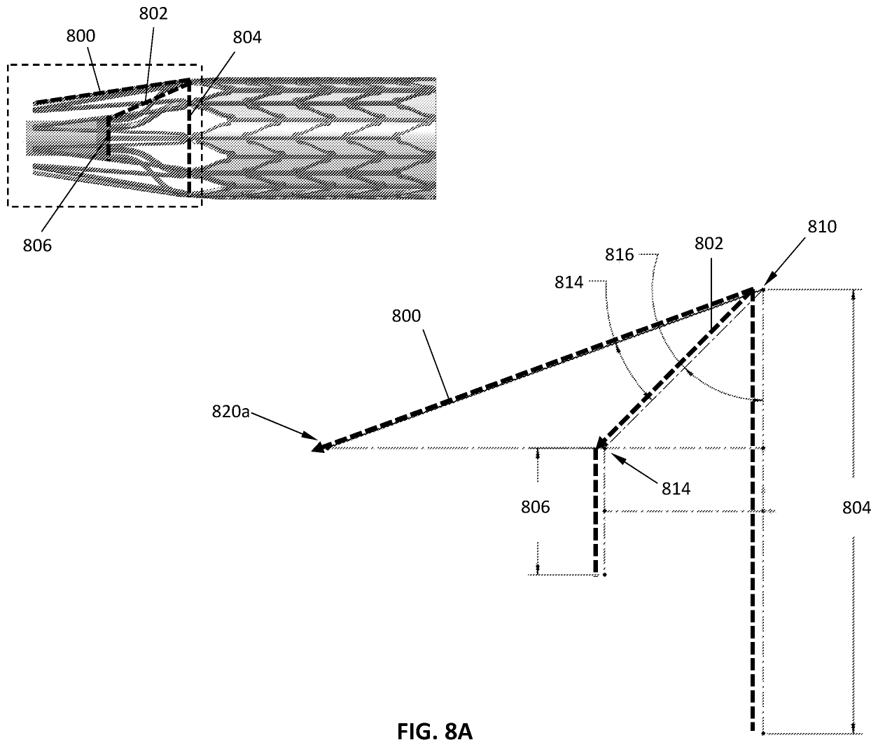

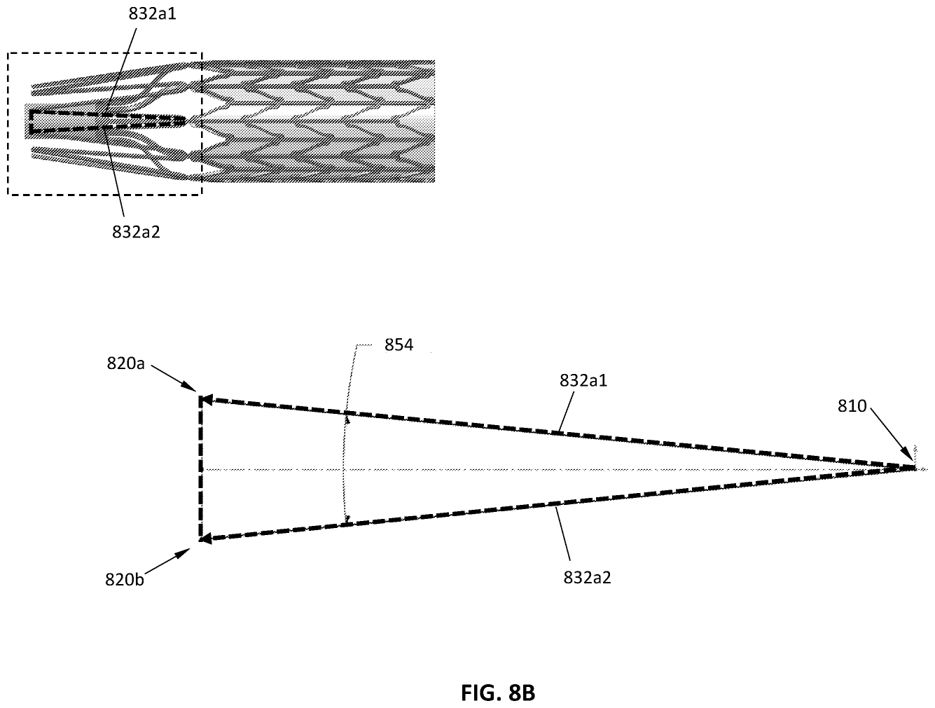

FIG. 8A illustrates exemplary dimensions (e.g., angles) defining aspects of the tension members 332a or 532a (FIGS. 3A-3B or FIGS. 5-7). A tension member line 800 may be measured from a point of connection 810 of the tension member along the blood conduit outer diameter 804 to a point of connection 820a of the tension member along the proximal shaft diameter 806. A strut line 802 may be measured from the point of connection 810 of the strut along the blood conduit outer diameter 804 to a point of connection 814 of the strut along the proximal shaft diameter 806. A tension member angle 814 may be measured between the tension member line 800 and the strut line 802. A strut angle 816 may be measured between the strut line 802 and the blood conduit diameter 804. In some examples, the tension member angle 814 plus the strut angle 816 is less than 90 degrees. In some examples, the tension member angle 814 is greater than or equal to 5 degrees. In some examples, the strut angle 816 is greater than or equal to 5 degrees.

FIG. 8B illustrates additional exemplary dimensions (e.g., angles) defining aspects of the tension members 332a or 532a (FIGS. 3A-3B or FIGS. 5-7). As described above, there are two tension members for each of the struts. Thus, each strut has an associated pair of tension members, for example, a first tension member 832a1 and a second tension member 832a2. The first tension member 832a1 may be measured from a point 810 along (or near) the blood conduit outer diameter (804 in FIG. 8A) to the point 820a along (or near) the proximal shaft outer diameter (806 in FIG. 8A) where the first tension member 832a1 attaches. The second tension member 832a2 may be measured from the point 810 along (or near) the blood conduit outer diameter (804 in FIG. 8A) to a point 820b along (or near) the proximal shaft outer diameter (806 in FIG. 8A) where the second tension member 832a2 attaches. In some examples, a span angle 854 between the first tension member 832a1 and the second tension member 832a2 may be less than or equal to 75 degrees. In some examples, a span angle 854 between the first tension member 832a1 and the second tension member 832a2 may be less than or equal to 60 degrees.

1. An intravascular blood pump, comprising:
a collapsible blood conduit having an inner lumen for passing blood therethrough, wherein a proximal portion of the blood conduit includes an impeller region configured to accommodate at least a portion of an impeller therein; and
a plurality of struts and one or more tension members extending proximally from the blood conduit and radially inward toward a shaft, wherein the plurality of struts are proximally coupled to the shaft at a first axial region along the shaft, and the one or more tension members is proximally coupled to the shaft at a second axial region along the shaft, wherein first axial region is at a different axial position along the shaft than the second axial region.

2. The intravascular blood pump of claim 1, wherein the plurality of struts and one or more tension members are configured to provide the impeller region resistance to lateral deflection.

3. The intravascular blood pump of claim 1, wherein the second axial region extends further proximally along the shaft relative to the first axial region.

4. The intravascular blood pump of claim 1, wherein the first axial region extends further proximally along the shaft relative to the second axial region.

5. The intravascular blood pump of claim 1, wherein at least a portion of the one or more tension members is radially outside of the plurality of struts.

6. The intravascular blood pump of claim 1, wherein the blood conduit includes an expandable scaffold comprising a pattern of axially and radially extending elements.

7. The intravascular blood pump of claim 6, wherein the blood conduit includes a membrane that covers the expandable scaffold.

8. The intravascular blood pump of claim 1, wherein the one or more tension members is fixedly coupled to the shaft at the second axial region.

9. The intravascular blood pump of claim 1, wherein the one or more tension members is coupled to the shaft by a tension control element that is configured to move axially relative to the shaft, wherein axial movement of the tension control element adjusts an amount of tension placed on the one or more tension members and an amount of lateral stiffness imparted to the impeller region of the blood conduit.

10. The intravascular blood pump of claim 9, wherein the tension control element includes a locking feature configured to lock an axial position of the tension control element relative to the shaft.

11. The intravascular blood pump of claim 1, wherein the one or more tension members includes a plurality of tension members.

12. The intravascular blood pump of claim 11, wherein the plurality of tension members are equally radially distributed around a proximal end of the blood conduit.

13. The intravascular blood pump of claim 11, wherein the plurality of tension members are equally radially distributed the shaft.

14. The intravascular blood pump of claim 1, wherein the plurality of struts are equally radially distributed around a proximal end of the blood conduit.

15. The intravascular blood pump of claim 1, wherein the plurality of struts are equally radially distributed around the shaft.

16. The intravascular blood pump of claim 1, wherein the plurality of struts are coupled to a hub of the shaft.

17. The intravascular blood pump of claim 1, wherein the one or more tension members is coupled to a hub of the shaft.

18. The intravascular blood pump of claim 1, wherein the plurality of struts are coupled at the same axial position along the shaft.

19. The intravascular blood pump of claim 1, wherein the plurality of struts are coupled along at different axial positions along the shaft.

20. The intravascular blood pump of claim 1, wherein the one or more tension members includes a plurality of tension members, wherein the plurality of tension members are coupled at the same axial position along the shaft.

21.-84. (canceled)
